(19) United States
(12) Patent Application Publication
(10) Pub. No.: US 2007/0128201 A1
(43) Pub. Date: Jun. 7, 2007

(54) Combined use of ecteinascidin-743 and platinum antineoplastic compounds

(75) Inventors: Maurizio D'Incalci, Milan (IT); Luca Gianni, Milan (IT); Raffaella Giavazzi, Milan (IT); Margarita Garcia Martin, Barcelona (ES); Ian Judson, London (GB); Jose Maria Jimeno Donaque, Madrid (ES); Cristiana Sessa, Bellinzona (CH)

Correspondence Address: KING & SPALDING, 1185 AVENUE OF THE AMERICAS, NEW YORK, NY, 10036-4003, US

(73) Assignee: Pharma Mar, S.A.U., Madrid (ES)

(21) Appl. No.: 10/558,133

(22) PCT Filed: Jun. 1, 2004
(86) PCT No.: PCT/GB04/02319

§ 371 Date: Nov. 15, 2006

(30) Foreign Application Priority Data
May 29, 2003 (GB) 0312407.0

(51) Int. Cl. A61K 39/395; A61K 33/24

(52) U.S. Cl. 424/155.1; 424/649

(57) ABSTRACT

ET-743 can be used to mitigate resistance to and potentiate the cytotoxic effects of a platinum coordination complex anti-neoplastic agent in a human cancer patient.

The invention relates to a treatment, more particularly an improved use of antitumoral compounds in cancer therapy.

### FIELD OF THE INVENTION

The present invention is directed to the use of ecteinascidin 743 and products containing this compound for cancer therapy, in particular to the use of ecteinascidin 743 in combination with an antineoplastic platinum coordination complex in the treatment of cancer.

### BACKGROUND OF THE INVENTION

Cancer comprises a group of malignant neoplasms that can be divided into two categories: carcinoma, comprising a majority of the cases observed in the clinics, and other less frequent cancers, which include leukemia, lymphoma, central nervous system tumours and sarcoma. Carcinomas have their origin in epithelial tissues while sarcomas develop from connective tissues and those structures that had their origin in mesoderm tissues. Sarcomas can affect, for instance, muscle or bone and occur in the bones, bladder, kidneys, liver, lung, parotid, spleen, etc.

Cancer is invasive and tends to metastasise to new sites. It spreads directly into surrounding tissues and also may be disseminated through the lymphatic and circulatory systems.

Many treatments are available for cancer, including surgery and radiation for localised disease, and drugs. However, the efficacy of available treatments on many cancer types is limited, and new, improved forms of treatment showing clinical benefit are needed. This is especially true for those patients presenting with advanced and/or metastatic disease. It is also true for patients relapsing with progressive disease after having been previously treated with established therapies for which further treatment with the same therapy is mostly ineffective due to acquisition of resistance or to limitations in administration of the therapies due to associated toxicities.

Chemotherapy plays a significant part in cancer treatment, as it is required for treatment of advanced cancers with distant metastasis and it is often helpful for tumor reduction before surgery. Many anti-cancer drugs have been developed based on various modes of action.

The most commonly used types of anticancer agents include: DNA-alkylating agents (e. g., cyclophosphamide, ifosfamide), antimetabolites (e. g., methotrexate, a folate antagonist, and 5-fluorouracil, a pyrimidine antagonist), microtubule disrupters (e. g., vincristine, vinblastine, paclitaxel), DNA intercalators (e. g., doxorubicin, daunomycin, cisplatin), and hormone therapy (e. g., tamoxifen, flutamide). The ideal antineoplastic drug would kill cancer cells selectively, with a wide therapeutic index relative to its toxicity towards non-malignant cells. It would also retain its efficacy against malignant cells, even after prolonged exposure to the drug. Unfortunately, none of the current chemotherapies possess an ideal profile. Most possess very narrow therapeutic indexes and, in practically every instance, cancerous cells exposed to slightly sublethal concentrations of a chemotherapeutic agent will develop resistance to such an agent, and quite often cross-resistance to several other antineoplastic agents.

Combination therapy using drugs with different mechanisms of action is an accepted method of treatment which helps preventing development of resistance by the treated tumor.

The ecteinascidins (herein abbreviated ET or ET's) are exceedingly potent antitumor agents isolated from the marine tunicate Ecteinascidia turbinata. Several ecteinascidins have been reported previously in the patent and scientific literature. See, for example U.S. Pat. No. 5,089,273, which describes novel compounds of matter extracted from the tropical marine invertebrate, Ecteinascidia turbinata, and designated therein as ecteinascidins 729, 743, 745, 759A, 759B and 770. These compounds are useful as antibacterial and/or antitumor agents in mammals. U.S. Pat. No. 5,478,932 describes ecteinascidins isolated from the Caribbean tunicate Ecteinascidia turbinata, which provide in vivo protection against P388 lymphoma, B16 melanoma, M5076 ovarian sarcoma, Lewis lung carcinoma, and the LX-1 human lung and MX-1 human mammary carcinoma xenografts.

One of them, ecteinascidin-743 (ET-743), is a novel tetrahydroisoquinoline alkaloid isolated from the marine ascidian Ecteinascidia turbinata that has considerable in vitro and in vivo antitumor activity in murine and human tumors, and is presently in clinical trials.

Potent antitumor activity has been demonstrated in a broad range of in vivo tumor models, including human tumor xenografts in nude mice. Et-743 has a novel complex mechanism of action at the level of gene transcription. ET-743 binds to guanine-cytosine rich sequences in the minor groove of DNA and alkylates guanine residues at the N2 position

An in vitro bone marrow assay using human, murine and canine progenitor cells, showed equal sensitivity of erythropoid and myeloid cells to ET-743. Prolonged or repeated exposure to the drug proved more toxic to hematopoietic progenitors than a single 1-hour exposure. The therapeutic index of ET-743 was more favourable with prolonged exposure.

A clinical development program of ET-743 in cancer patients was started with phase I studies investigating 1-hour, 3-hour, 24-hour and 72-hour intravenous infusion schedules and a 1 hour daily×5 (d×5) schedule. In Phase I and II clinical trials ET-743 has shown significant antitumor activity against several human malignancies including soft tissue sarcomas and ovarian carcinomas. Further detail on the use of ET-743 for the treatment of the human body for cancer is given in WO 0069441, incorporated herein by specific reference.

A well known and used family of anticancer agents are the platinum compounds. Cisplatin (cis-diaminedichloroplatinum (II)) is a platinum coordination complex first identified in 1965 as a cytotoxic agent. It has broad activity as an antineoplastic agent and is especially useful in the treatment of epithelial malignancies. Other platinum coordination complexes that have been evaluated in clinical trials include carboplatin, tetraplatin, ormiplatin, iproplatin and oxaliplatin.

The treatment of cancer patients with platinum coordination complex antineoplastic agents, such as cisplatin or carboplatin has increased substantially in the last decade. Cisplatin has proved to be useful in the treatment of multiple malignancies including testicular cancer, ovarian cancer, and small cell lung cancer, whereas carboplatin has proved to be useful in brain tumors, endometrial cancer, germ cell tumors and head and neck cancer. The mechanism of action is currently unknown but may be related to the ability of these compounds to bind to DNA and form various types of inter- and intrastrand crosslinks that possibly interfere with both DNA and RNA synthesis.

Cancer patients eventually become resistant to treatment with platinum coordination complexes, such as cisplatin or carboplatin. The mechanism of resistance to these compounds is unclear but may be related to decreased drug accumulation, elevation of intracellular concentrations of metallothioneines or glutathione which bind and inactivate the drug, or to decreased drug-DNA adduct formation or repair. Therefore there is a need to develop effective therapies that overcome this resistance.

In cancer cell lines growing in vitro the combination of ET-743 and cisplatin showed an additive or synergistic effect evaluated by isobologram analysis. This synergistic effect has also been confirmed in vivo: Erba, E. et al. “ET-743 and cisplatin (DDP) show in vitro and in vivo synergy against human sarcoma and ovarian carcinoma cell lines”, Proceed. AACR-NCI-EORTC November 2001, abstract 406; Faircloth, Glynn Thomas, Jr., et al. “In vivo combinations of chemotherapeutic agents with Ecteinascidin 743 (Et743) against solid tumors”, Proceed. AACR-NCI-EORTC November 2001, abstract 387; D'Incalci M. et al.: “The combination of ET-743 and cisplatin (DDP): From a molecular pharmacology study to a phase I clinical trial.”, proceed. AACR March 2002, abstract 404; D'Incalci, M. et al. “In Human tumor xenografts the resistance to ET-743 or to cisplatin can be overcome by giving the two drugs in combination.”, Proceed. AACR-NCI-EORTC, November 2002, abstract 97.

The combination therapy comprising ET-743 is also the object of WO 02 36135, incorporated herein by specific reference in its entirety.

It is an object of the invention to provide efficacious methods and products for preventing resistance or overcoming established resistance to platinum coordination complex anti-neoplastic agents in human patients. It is another object of the invention to provide an effective method and products for potentiating the cytotoxic effects of platinum coordination complex anti-neoplastic agents in the clinical setting.

### SUMMARY OF THE INVENTION

Unexpectedly, we found that when given in combination, the maximum dosage of ET-743 and a platinum compound, particularly cisplatin or carboplatin, can be given without an increase or addition of the toxicity. This has been confirmed in the clinical trials, in which full dose of cisplatin and carboplatin has been given successfully with escalating doses of Et-743.

Thus, the subject invention concerns a novel treatment regimen for cancer patients whereby the platinum compound is administered in combination with ET-743.

The invention further provides a method for treatment of a human cancer patient which involves administering a platinum compound and ET-743, in which the amount of the platinum compound is at least 50%, at least 75%, at least 85%, at least 90%, at least 95%, or at least 100% of the Recommended Dose for the platinum compound in the absence of ET-743, and the amount of the ET-743 is at least 50%, at least 75%, at least 85%, at least 90%, at least 95%, or at least 100% of the Recommended Dose for ET-743 in the absence of the platinum compound. The Recommended Doses are based on studies of Dose Limiting Toxicity. Preferably the amounts of the platinum compound and ET-743 are both at least at least 85%, at least 90%, at least 95%, or at least 100% of the respective Recommended Dose.

In another aspect the present invention is directed to the use of ET-743 in the preparation of a medicament for an effective treatment of a human cancer patient by combination therapy employing ET-743 with a platinum compound, characterised in that the combination overcomes resistance to platinum anti-neoplastic compounds without increasing the toxicity of each drug.

In a related aspect, the invention provides a method of treating a human cancer patient with a platinum compound, wherein ET-743 is administered as a combination therapy without a compensating drop in the dose of the platinum compound.

In yet another embodiment of the present invention, there is provided a method of reducing resistance to platinum anti-neoplastic compounds in an individual having a neoplastic disease comprising administering to an individual ET-743 and the platinum compound in a dosage range which is the same as the dosage given if each of ET-743 and the platinum compound were administered alone.

The present invention also provides a pharmaceutical composition containing a recommended dose of ET-743 for weekly administration in combination with a platinum compound and a pharmaceutically acceptable carrier.

In a further aspect of the present invention, a medical kit for administering ET-743 in combination with an antineoplastic platinum compound is provided, comprising printed instructions for administering ET-743 according to the dosing schedules set forth below, and a supply of ET-743 in dosage units for at least one cycle, wherein each dosage unit contains the appropriate amount of ET-743 for the treatments as defined above and a pharmaceutically acceptable carrier.

### DETAILED DESCRIPTION

ET-743 is a natural compound represented by the following formula:

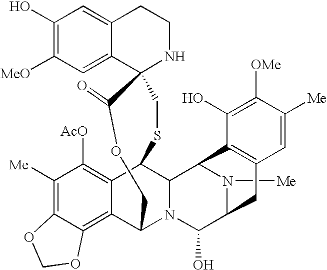

The term “ET-743” is also intended here to cover any pharmaceutically acceptable salt, ester, solvate, hydrate or any other compound which, upon administration to the recipient is capable of providing (directly or indirectly) the compound as described herein. However, it will be appreciated that non-pharmaceutically acceptable salts also fall within the scope of the invention since these may be useful in the preparation of pharmaceutically acceptable salts. The preparation of salts and prodrugs and derivatives can be carried out by methods known in the art.

ET-743 is supplied and stored as a sterile lyophilized product, consisting of ET 743 and excipient in a formulation adequate for therapeutic use.

The combinations of the present invention comprise ET-743 and an antineoplastic platinum compound, preferably a coordination complex. Preferred complexes include cisplatin, carboplatin, tetraplatin, ormiplatin, iproplatin, oxaliplatin and the like. Preferably, the platinum coordination complex is cisplatin or carboplatin, more preferably cisplatin.

The two drugs can be given simultaneously or one after the other in either sequence, preferably in a sequence.

As it has been said, the invention provides a method for treatment a human cancer patient. Preferably the patients are relapsing or refractory to previous chemotherapy. Most preferably the patients are ovarian cancer, head and neck cancer, NSCL carcinoma or melanoma patients. In a particularly preferred embodiment the patients are ovarian cancer patients and the previous therapy comprises treatment with platinum compounds.

Additionally, the present invention provides a method of treating cancer in humans, comprising a step of intravenously infusing a composition comprising ET-743 into a human having cancer at continuous dosage over a period up to 4 hours, followed or preceded by intravenously infusing a composition comprising a platinum antineoplastic compound into a human having cancer at a continuous dosage wherein the step of infusing is repeated weekly on a cyclic basis.

The infusing step is typically repeated on a cyclic basis. The cyclic basis comprises two phases, the phase of weekly infusing and a phase of not infusing, referred to as a rest phase. In the rest phase the patients are allowed to recover. Usually the cycle is worked out in weeks, and thus the cycle comprises one or more weeks of an infusion phase, and one or more weeks of a rest phase. The rest period can be longer or shorter than the infusion phase. The preferred duration of each cycle is of 2 to 4 weeks; multiple cycles can be given as needed. Cycles of 3 or 4 weeks with 1 or 2 weeks infusion is most preferred.

When ET-743 is administered in combination with cisplatin, the dosage amount of ET-743 is preferably below 700 μg/m²/day on a day 1 & 8 every 3 or 4 weeks schedule, preferably from about 400 to about 650 μg/m²/day, more preferably from about 500 to about 650 μg/m²/day, even more preferably from about 550 to about 650 μg/m²/day. In this case the schedule most preferred is the administration of both compounds on a day 1 & 8 every 4 weeks.

On the other hand, when ET-743 is administered in combination with carboplatin, the dosage of ET-743 is preferably below 1200 μg/m²/day on a day 1 every 3 weeks schedule, preferably between 650 and 1200 μg/m²/day, more preferably between 800 and 1000 μg/m²/day, even more preferably between 800 and 900 μg/m²/day.

The dosage amount of cisplatin is the full dosage range used according to the type of schedule given. Preferably it is about 30-60 mg/m²/day, more preferably about 40-50 mg/m²/day, even more preferably about 40 mg/m²/day.

The dosage amount of carboplatin is the full dosage range used according to the type of schedule given. Preferably it is about 200-400 mg/m²/day, more preferably about 250-300 mg/m²/day.

In a particular embodiment, the infusion time of ET-743 is between 1 and 3 hours, preferably between 2 and 3 hours. Especially preferred is a time of about 3 hours.

The above schedules and dosages allow for an effective combination cancer therapy in humans, while avoiding toxicities. We have found that ET-743 in combination with cisplatin or carboplatin is effective in the treatment of several cancer types, including advanced or metastatic. Preferably, the combination ET-743 with a platinum compound is used according to the above schedules and dosages for the treatment of sarcoma, osteosarcoma, ovarian cancer, breast cancer, melanoma, head and neck cancer, colorectal cancer, mesothelioma, renal cancer, endometrial cancer and lung cancer.

Depending on the type of tumour and the developmental stage of the disease, the treaments of the invention are useful in preventing the risk of developing tumours, in promoting tumour regression, in stopping tumour growth and/or in preventing metastasis.

Although guidance for the dosage is given above, the correct dosage of the compound will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

### EXAMPLES

### Example 1

In order to evaluate the effects of the combination of ET-743 and cisplatin (DDP) in vivo we selected some xenografts relatively resistant to a single dose of DDP and moderately sensitive to a single dose of ET-743. For the administration of the drugs appropriate vehicles were injected, using the same schedule and route of injection as the drug therapies. ET-743 and DDP were given 1 h apart in sequence or simultaneously. In xenografts transplanted s.c. tumor growth was monitored and tumor weight (TW) was determined by measuring the tumor diameters with a Vernier caliper every 2-4 days and using the formula TW=d²×D/2 (where d and D represent the shortest and the longest diameter respectively).

The maximal single i.v. dose of DDP and ET-743 that caused no toxic death were respectively 12 mg/Kg and 0.2 mg/Kg. The same dose of each drug could be given when the two drugs were administered in combination with a tolerable toxicity, with a maximal weight loss ranging from 10 to 26% in different experiments(n=14) with a median value of 15%. Surprisingly, the treatment with the combination caused only a slightly higher weight loss than the treatment with each drug alone. The toxicity did not appear different when the two drugs were given simultaneously or one given after the other with an interval of 1 h in either of the two sequences.

The antitumor activity of the combination was greater than that of each drug alone in all models. In TE-671 rhabdomyosarcoma and SK-N-DZ neuroblastoma all three combinations (i.e. ET-743 given 1 h before DDP or at the same time or 1 h after DDP) were compared and no significant differences in the antitumor activity were observed. Also in H & N FADU, in NSCLC LX-1, in melanoma H-187 and in the ovarian SKOV where the two sequences were compared no consistent differences related to the sequence was found.

Collectively all the data indicate that the antitumor activity of the combination was greater than that of each drug alone and the sequence does not influence the treatment efficacy and toxicity in a consistent fashion.

### Example 2

The observation that the toxicity of the combination appeared very modest prompted us to test the effect of the combination of ET-743 and DDP splitting the dose of both drugs in three administrations with an interval of 4 days.

1A9 ovarian carcinoma xenografts were relatively resistant to the two drugs used as monotherapy. In contrast DDP at 4 mg/kg (Q4×3) for a total dose of 12 mg/kg given simultaneously to ET-743 at 0.1 mg/kg (Q4×3) for a total dose of 0.3 mg/kg induced a significant TWI of 73%.

Again we observed no toxic deaths or severe toxicity with the combination (mean body weight loss 16%) compared to the single drugs (14% and 12% with ET-743 and DDP respectively).

Thus the combination therapy allows high dosages and even in tumors in which the two drugs produced no significant activity given alone there was evidence of activity of the combination of each of the drugs. The combination is particularly successful in overcoming resistance in ovarian carcinoma xenograft.

### Example 3

In ovarian carcinoma patients the tumor spreads into the peritoneal cavity. Therefore to mimic clinical disease we have selected a human ovarian xenograft, HOC 8, which was transplanted intraperitoneally from ascites and disseminated in the peritoneal cavity. This tumor is partially sensitive to DDP (ILS=139%) and insensitive to ET-743 (ILS=21% and 23% with 0.05% and 0.15 mg/kg, Q4×3).

When the two drugs were combined the effect was much greater than that of each drug given as single agent with a dramatic increase in survival. Both the low (ILS=258% versus vehicle) and the high (ILS=322% versus vehicle) dose of ET-743 combined with DDP increased the survival time of mice bearing HOC8, that was significantly improved compared to DDP as monotherapy (ILS=49% and 76% versus DDP with low and high dose of ET-743 respectively). Three animals were still alive after 12 months, two of them belonging to the group receiving the high ET-743 dose. They were sacrificed and a detailed macroscopic and microscopic pathological evaluation was performed. The mouse belonging to the group receiving the low ET-743 dose was apparently cured as the microscopic analysis of liver, splen, pancreas, bone marrow, diagram, ovary uterus omentum and several lymphnodes were negative. Instead both the other long term surviving mice showed a residual tumor at the level of omentum and in one of them a single metastasis in the uterus was found, while in the other organs no metastases were detected.

This example shows the potential of the combination in ovarian cancer, even if there is metastasis.

### Example 4

We designed a multicenter dose finding trial, on a day 1 & 8 every 3 weeks schedule, with escalating doses of ET given as a 3-hr infusion with steroids and antiemetic prophylaxis, followed 30 minutes later by 1 hr infusion cisplatin at a fixed dose of 40 mg/m², with 2 L NS hydration.

36 patients were entered (15 with ovarian cancer, 6 with uterus cancer, 14 with soft tissue sarcoma, 1 with other tumor type). Prior treatments of the patients were the following:

PRIOR TREATMENTS

| | |
|---|---|
| No OF PTS WITH PRIOR CHEMO FOR | 35 |
| ADVANCED DISEASE | |
| N° OF PRIOR CHEMO REGIMENS | median 1 |
| FOR ADVANCED DISEASE | range 0-2 |
| N° OF PTS W/PRIOR PLATINUM | 22 (61%) |
| RESPONDERS | 9 |
| RESISTANT | 13 |
| N° OF PTS W/PRIOR CARBOPLATIN | 16 |
| RESPONDERS | 6 |
| RESISTANT | 10 |

ET-743 doses levels were 300, 400, 500, 600 and 700 μg/m²/day; 3-6 were treated per dose level according to toxicity.

ET-743 escalation was uneventful until 500 μg/m²; at 600 μg/m² patients were accrued in 2 separate risk cohort according to prior chemotherapy extent: low risk 1 regimen (LR); high risk ≧2 regimens (HR).

The following table illustrates the hematologial toxicity found:

HEMATOLOGICAL TOXICITY

| ET-743 | | | | |
|---|---|---|---|---|
| D 1&8 | Enrolled | Treated | Pts With toxicity at cy 1 | |
| μg/m² | pts | pts | G3 | G4 |
| 300 | 3 | 3 | none | none |
| 400 | 3 | 4*^{#} | none | none |
| 500 | 8 | 7° | 2 ANC | none |
| 600 | 15 | 15° | 8 ANC | none |
| 700 | 7 | 7 | 2 ANC 1 Hb | 2 ANC 1 PLT |

*7 days duration of G4 neutropenia → DLT
°1 pt at 500 and 1 pt at 600 failed to recover from neutropenia by >d35 → DLT
^{#}1 pt enrolled at 500 received 400 at cycle 1

The following table illustrates the non-hematologial toxicity found:

NON-HEMATOLOGICAL TOXICITY
BY DOSE & SCHEDULE

| ET-743 | N° | | | | | | | |
|---|---|---|---|---|---|---|---|---|
| dose | of | | | | | | | |
| D 1&8 | treated | Asthenia | | N&V | | | AST/ALT | |
| μg/m² | pts | G1 | G2 | G1 | G2 | G3 | ≦G2 | G3 |
| 300 | 3 | 1 | 1 | — | — | — | 1 | — |
| 400 | 4 | 1 | 1 | — | 1 | — | 2 | 1 |
| 500 | 7 | 5 | 1 | 4 | 1 | — | 6 | — |
| 600 | 15 | 6 | — | 12 | 1 | — | 8 | 4 |
| 700 | 7 | 4 | 1 | 2 | 3 | 1 | 5 | 2 |

The following table illustrates other non-hematologial toxicity found:

OTHER NON-HEMATOLOGICAL TOXICITY
BY DOSE & SCHEDULE

| ET-743 dose | | | |
|---|---|---|---|
| D 1&8 μg/m² | Treated Pts | G1 | G2 |
| 300 | 3 | | |
| 400 | 4* | | 1 anorexia |
| | | | 1 phlebitis |
| 500 | 7* | | |
| 600 | 15 | 3 SNP | 1 abdo cramps |
| | | | 1 abdo pain |
| 700 | 7 | | 1 anorexia |

Dose Limiting Toxicities (DLTs):
500: 1/7 treated pts failed to recover at day 35
600: 3/15 treated pts
   1 failure to recover hematological toxicity at day 35
   1 ALT grade 3 not recovered to B/L
   1 failure to retreat on day 8 bilirubin grade 1, ALT grade 3
700: 2/7 treated pts ANC grade 4 lasting >7 days (1 pt also had concomitant Gr 4 thrombocytopenia and failure to recover hematological toxicity at day 35)

The following table illustrates the observed efficacy:

| | | | ET-743 | | | |
|---|---|---|---|---|---|---|
| | | | dose | | | |
| | | | D 1&8 | SITES OF | | TTP |
| TUMOR | PRIOR TX | | μg/m² | DISEASE | BEST RESP | mos |
| Ovary | carboplatin + taxol | NE | 600 | Pelvis, liver | PR | 6+ |
| | paclitaxel | PD | | | | |
| STS gyn | adriamycin/ifosfamide | AD | 700 | Lung | PR | 3+ |
| Ovary | carboplatin + taxol | NC | 400 | Lung | PR | 6 |
| | topotecan | PD | | | | |
| Ovary | carboplatin + taxol | PR | 600 | Liver Abdomen | Unc PR | Too |
| | | | | | | early |
| STS- | epirubicin + | AD | 500 | Abdomen | Radiological | 5+ |
| gyn | ifosfamide | | | | PR non | |
| | gemcitabine | PD | | | measurable | |
| | | | | | lesions | |
| Uterus | carboplatin | NE | 400 | Bone, Pelvis | Radiological | 1 |
| Colon | taxol + epirubicin + | CR | | Lung | PR non | |
| | cisplatin | | | | measurable | |
| | | | | | lesions | |

(PR: partial response; PD: progressive disease; CR: complete response; NC: no change; AD: adjuvant; NE: non-evaluable; TTP: time to progression)

From this study we concluded that:
In this population the MTD is 700 μg/m² in previously treated patients day 1 & 8 every 4 weeks
Recommended dose (RD) in previously treated patients is 500 μg/m² day 1 & 8 every 4 weeks
The DLT is myleosuppression, particulatly neutropenia
At doses ≧600 μg/m² day 1 & 8 every 3 weeks delayed recovery from neutropenia was observed in the majority of patients.
Main non-hematological toxicities are dose-dependent nausea and vomits (N&V), Asthenia and Liver toxicity (always reversible and mild up to 600 μg/m²/day).
Main non-hematological toxicities were dose-dependent N&V and asthenia
Optimum interval of re-treatment is day 28

### Example 5

We designed a multicenter dose finding trial, on a day 1 every 3 weeks schedule, with carboplatin given at a fixed dose of 300 mg/m² as a 1 hour infusion followed by escalating doses of ET given as a 3 hours infusion with steroids and antiemetic prophylaxis.

11 patients were entered (6 with ovarian cancer, 1 with lung cancer, 4 with soft tissue sarcoma). Prior treatments of the patients were the following:

| | | |
|---|---|---|
| N of patients | 11 | |
| Type of tumor | | |
| Non Small Cell Lung Cancer (NSCLC) | 1 | |
| Epithelial Ovarian Carcinoma | 6 | |
| Soft tissue sarcoma | 4 | |
| Previous treatment | | |
| One line chemotherapy | 6 | |
| Two or more lines | 5 | |
| Previous treatment with Carboplatin | 6 | (all Ovarian Carcinoma) |

ET-743 doses levels were 500, 650 and 800 μg/m²/day; 3-6 pts were treated per dose level according to toxicity.

The maximum tolerated dose (MTD) was defined as the highest dose level tested of the combination at which at least 2 patients experience a DLT in cycle 1. If one patient encountered drug-induced DLT during either cycle 1 or 2, up to a maximum of 6 patients could be treated at that level. If DLT was not observed in the additional patients, new patients could be treated at the next higher dose level.

The cycle 1 haematological toxicities for platelets and absolute neutrophil count (ANC) are reported in the following table:

| | No of | Neutropenia | | | | Thrombopenia | | | |
|---|---|---|---|---|---|---|---|---|---|
| Level | patients | G0 | G1 | G2 | G3 | G0 | G1 | G2 | G3 |
| 500 | 3 | 2 | 0 | 1 | 0 | 0 | 2 | 1 | 0 |
| 650 | 3 | 0 | 1 | 2 | 0 | 0 | 3 | 0 | 0 |
| 800 | 5 | 2 | 1 | 1 | 1 | 1 | 1 | 1 | 2 |

Two patients developed DLT during the first course with grade 3 thrombocytopenia in dose level 3. Both patients had Ovarian Carcinoma pretreated with Carboplatin.

The following table shows haematological toxicities for platelets and ANC for all the courses administered, as well as the number of cycles without haematological recovery by day 21 and 28.

| | | No of courses | No of courses |
|---|---|---|---|
| | | without | without |
| | | haematological | haematological |
| ET-743 dose | No of | recovery | recovery at |
| administration | courses | at day +21 | day +28 |
| 400* | 4 | 1/4 | 0/3 |
| 500 | 13⁽¹⁾ | 6/13 | 5/13 |
| 650 | 4⁽²⁾ | 4/4 | 0/3 |
| 800 | 9 | 3/6 | 0/5 |

*all after dose reduction;
⁽¹⁾8 after dose reduction;
⁽²⁾1 after dose reduction

| | Neutropenia | | | | Thrombopenia | | | |
|---|---|---|---|---|---|---|---|---|
| ET-743 dose administration | G0 | G1 | G2 | G3 | G0 | G1 | G2 | G3 |
| 400* | 0 | 2 | 2 | 0 | 0 | 3 | 1 | 0 |
| 500 | 4 | 3 | 5 | 1 | 5 | 5 | 2 | 1 |
| 650 | 0 | 1 | 3 | 0 | 0 | 3 | 0 | 1 |
| 800 | 4 | 1 | 1 | 3 | 3 | 2 | 1 | 3 |

*all after dose reduction; (1) 8 after dose reduction; (2) 1 after dose reduction

And the number of patients with dose delayed (and reduced) at cycle 2 with the reason for this delay is reported in the following table for each dose level:

| | No of patients receiving | Dose delayed (and | | |
|---|---|---|---|---|
| level | Cy 2 | reduced) at cycle 2 | | Reason |
| 500 | 3/3 | 1 | pt | ANC G2 |
| 650 | 3/3 | 3 | pts | Thrombopenia G1 |
| | | | | ANC G2 |
| | | | | ANC G2 |
| 800 | 4/5* | 2 | pts | ANC G3 |
| | | | | ANC G3 |

*one patient too early

As can be inferred from the cycle 1 data from 11 patients treated:
In this population the MTD is 800 μg/m² of ET-743 with carboplatin at a fixed target (300 mg/m²)
DLTs consist of Thrombocytopenia grade 3
In this population at the second dose level, 100% of patients had a dose delay with dose reduction at the second cycle because of haematological toxicity
At the third dose level, 50% of patients had a dose delay with dose reduction at the second cycle because of haematological toxicity

Given this haematological safety profile, with long lasting, though moderate neutropenia that prevent from achieving an adequate dose intensity of ET-743, and the 2 DLTs consisting of Thrombopenia grade 3 in two patients with Ovarian Carcinoma previously pretreated with carboplatin, it can be inferred the following schedule:
In patients previously treated with carboplatin: Administration of carboplatin at a fixed target (250 mg/m²) over 1 h infusion followed by ET-743 over 3 hours iv infusion on day 1 every 3 weeks.
In patients not previously treated with carboplatin: Administration of Carboplatin at a fixed target (300 mg/m²) over 1 h infusion followed by ET-743 over 3 hours iv infusion on day 1 every 3 weeks.

1. A method for mitigating resistance to a platinum coordination complex anti-neoplastic agent in a human cancer patient which comprises administering to the patient a platinum coordination complex anti-neoplastic agent and ET-743.

2. A method for potentiating the cytotoxic effects of a platinum coordination complex anti-neoplastic agent in a human cancer patient which comprises administering to the patient a platinum coordination complex anti-neoplastic agent and ET-743.

3. A method according to claim 1 or 2, wherein ET-743 is administered as a combination therapy with the platinum anti-neoplastic agent without a compensating drop in the dose of the platinum anti-neoplastic agent.

4. A method according to claim 1 or 2, which comprises administering to an individual the platinum anti-neoplastic agent and ET-743 in dosage amounts which are in the same range as the dosage given if each of ET-743 and the platinum compound is administered alone.

5. A method according to claim 4, wherein the amounts of the platinum anti-neoplastic agent and ET-743 are both at least 90% of the respective Recommended Dose.

6. A method according to claim 1, wherein the platinum anti-neoplastic agent is selected from cisplatin, carboplatin, tetraplatin, ormiplatin, iproplatin, oxaliplatin.

7. A method according to claim 6, wherein the platinum anti-neoplastic agent is cisplatin or carboplatin.

8. A method according to claim 7, wherein the platinum anti-neoplastic agent is cisplatin.

9. A method according to claim 1, wherein the platinum anti-neoplastic agent and ET-743 are administered sequentially.

10. A method according to claim 9, which comprises a step of intravenously infusing a composition comprising ET-743 into the patient at a continuous dosage over a period up to 4 hours, followed or preceded by intravenously infusing a composition comprising a platinum anti-neoplastic agent into the patient at a continuous dosage, and repeating weekly the steps of infusing on a cyclic basis.

11. A method according to claim 10, in which the cyclic basis comprises two phases, a phase of weekly infusing, referred to as an infusion phase, and a phase of not infusing, referred to as a rest phase, with the cycle comprising one or more weeks of an infusion phase, and one or more weeks of a rest phase.

12. A method according to claim 1, in which the patient is relapsing or refractory to previous chemotherapy.

13. A method according claim 1, in which the patient has a cancer selected from sarcoma, osteosarcoma, ovarian cancer, breast cancer, NSCL carcinoma, melanoma, head and neck cancer, colorectal cancer, mesothelioma, renal cancer, endometrial cancer and lung cancer.

14. A method according to claim 1, in which the patient has a cancer selected from ovarian cancer, NSCL carcinoma, melanoma, head and neck cancer.

15. A pharmaceutical composition for weekly administration containing a recommended dose of a platinum coordination complex anti-neoplastic agent and a recommended dose of ET-743.

16. The use of a platinum coordination complex anti-neoplastic agent in the preparation of a medicament for a method according to claim 1.

17. The use of ET-743 in the preparation of a medicament for a method according to claim 1.

18. A medical kit for administering an anti-neoplastic platinum anti-neoplastic agent in combination with ET-743, comprising a supply of ET-743 in dosage units for at least one cycle, wherein each dosage unit contains the appropriate amount of ET-743 and a pharmaceutically acceptable carrier, and printed instructions for administering ET-743 according to a dosing schedule.
